(19) United States
(12) Patent Application Publication
(10) Pub. No.: US 2014/0276052 A1
(43) Pub. Date: Sep. 18, 2014

(54) ABLATION CATHETER WITH ULTRASONIC LESION MONITORING CAPABILITY

(71) Applicants: Philips Koninklijke Electronics N.V., Eindhoven (NL); Boston Scientific Scimed, Inc., Maple Grove, MN (US)

(72) Inventors: Darrell L. Rankin, Milpitas, CA (US); Reena Pathania, Fremont, CA (US); Szabolcs Deladi, Veldhoven (NL); Dennis D. Clark, Lewistown, PA (US)

(73) Assignees: Philips Koninklijke Electronics N.V., Eindhoven (NL); Boston Scientific Scimed, Inc., Maple Grove, MN (US)

(21) Appl. No.: 14/210,725

(22) Filed: Mar. 14, 2014

Related U.S. Application Data
(60) Provisional application No. 61/852,459, filed on Mar. 15, 2013.

(51) Int. Cl. A61B 8/00; A61B 18/14; A61B 8/12

(52) U.S. Cl. CPC A61B 8/445; A61B 8/12; A61B 18/1492
USPC 600/439

(57) ABSTRACT

An ablation probe for treating and imaging body tissue includes an ablation electrode tip including an ablation electrode configured for delivering ablation energy to body tissue. A plurality of acoustic openings are disposed through the ablation electrode tip. A plurality of ultrasonic imaging sensors are positioned inside the ablation electrode tip. The ultrasonic imaging sensors are configured to transmit ultrasonic waves through the acoustic openings. A plurality of flex circuits are each electrically connected to one of the plurality of ultrasonic imaging sensors.

### CROSS-REFERENCE TO RELATED APPLICATION

This application claims priority to U.S. Provisional Application 61/852,459, filed Mar. 15, 2013, which is herein incorporated by reference in its entirety.

### TECHNICAL FIELD

The present disclosure relates generally to devices and systems for imaging tissue within the body during an ablation procedure. More specifically, the present disclosure relates to an ablation probe with ultrasonic imaging capabilities.

### BACKGROUND

In ablation therapy, it is often necessary to determine various characteristics of body tissue at a target ablation site within the body. In interventional cardiac electrophysiology (EP) procedures, for example, it is often necessary for the physician to determine the condition of cardiac tissue at a target ablation site in or near the heart. During some EP procedures, the physician may deliver a mapping catheter through a main vein or artery into an interior region of the heart to be treated. Using the mapping catheter, the physician may then determine the source of a cardiac rhythm disturbance or abnormality by placing a number of mapping elements carried by the catheter into contact with the adjacent cardiac tissue and then operate the catheter to generate an electrophysiology map of the interior region of the heart. Once a map of the heart is generated, the physician may then advance an ablation catheter into the heart, and position an ablation electrode carried by the catheter tip near the targeted cardiac tissue to ablate the tissue and form a lesion, thereby treating the cardiac rhythm disturbance or abnormality. In some techniques, the ablation catheter itself may include a number of mapping electrodes, allowing the same device to be used for both mapping and ablation.

Various ultrasound-based imaging catheters and probes have been developed for directly visualizing body tissue in applications such as interventional cardiology, interventional radiology, and electrophysiology. For interventional cardiac electrophysiology procedures, for example, ultrasound imaging devices have been developed that permit the visualization of anatomical structures of the heart directly and in real-time. In some electrophysiology procedures, for example, ultrasound catheters may be used to image the intra-atrial septum, to guide transseptal crossing of the atrial septum, to locate and image the pulmonary veins, and to monitor the atrial chambers of the heart for signs of a perforation and pericardial effusion.

Many ultrasound-based imaging systems comprise an imaging probe that is separate from the mapping and ablation catheters used to perform therapy on the patient. As a result, a position tracking system is sometimes used to track the location of each device within the body. In some procedures, it may be difficult for the physician to quickly and accurately determine the condition of tissue to be ablated. Moreover, the images obtained using many ultrasound-based imaging systems are often difficult to read and understand without reference to images obtained from a separate imaging system such as a fluoroscopic imaging system.

### SUMMARY

The present disclosure relates generally to devices and systems for imaging anatomical structures within the body during an ablation procedure.

In Example 1, an ablation probe for treating and imaging body tissue, the ablation probe comprising an ablation electrode tip, an ultrasonic imaging sensor, and a flex circuit. The ablation electrode tip includes an ablation electrode configured for delivering ablation energy to body tissue. The ultrasonic imaging sensor is disposed within the ablation electrode tip and is configured to transmit and receive ultrasonic waves. The flex circuit is mechanically and electrically connected to the ultrasonic imaging sensor.

In Example 2, the ablation probe of Example 1, further comprising a plurality of ultrasonic imaging sensors and a plurality of flex circuits. The plurality of ultrasonic imaging sensors is disposed within the ablation electrode tip, and each of the plurality of ultrasonic imaging sensors is configured to transmit and receive ultrasonic waves. The plurality of flex circuits are each mechanically and electrically connected to one of the plurality of ultrasonic imaging sensors.

In Example 3, the ablation probe of Example 2, and further comprising a plurality of electrical conduits, each electrically connected to one of the plurality of ultrasonic imaging sensors via one of the plurality of flex circuits.

In Example 4, the ablation probe of either of Examples 2 or 3, wherein the plurality of ultrasonic imaging sensors comprises at least three ultrasonic imaging sensors, wherein the plurality of flex circuits comprises at least three separate and distinct flex circuits each connected to one of the ultrasonic imaging sensors.

In Example 5, the ablation probe of any of Examples 2-4, wherein the plurality of ultrasonic imaging sensors are each mounted on one of the plurality of flex circuits within the ablation electrode tip.

In Example 6, the ablation probe of any of Examples 2-5, wherein each of the plurality of flex circuits has a proximal end terminating within a central bore of the ablation electrode tip.

In Example 7, the ablation probe of any of Examples 2-6, wherein the ablation electrode tip has a tubular electrode shell and a plurality of acoustic openings disposed therein, and wherein each of the ultrasonic imaging sensors is aligned with a respective one of the acoustic openings.

In Example 8, the ablation probe of any of Examples 2-7, wherein the plurality of ultrasonic imaging sensors includes three ultrasonic imaging transducers oriented circumferentially about the ablation electrode tip.

In Example 9, the ablation probe of Example 7, wherein the ablation tip further comprises a plurality of irrigation ports formed in the tubular electrode shell distally of the acoustic openings.

In Example 10, an ablation probe for treating and imaging body tissue, the ablation probe comprising an ablation electrode tip, a plurality of acoustic openings in the tip, a plurality of ultrasonic imaging sensors, and a plurality of acoustic cups. The ablation electrode tip includes an ablation electrode configured for delivering ablation energy to body tissue, and the plurality of acoustic openings are disposed through the ablation electrode tip. The plurality of ultrasonic imaging sensors are positioned inside the ablation electrode tip, each aligned with one of the acoustic openings. Each of the plurality of acoustic cups covers one of the ultrasonic imaging sensors.

In Example 11, the ablation probe of Example 10, wherein each of the acoustic cups comprises a main cup section, a back step extending from a side of the main cup section.

In Example 12, the ablation probe of Example 11, wherein the main cup section is positioned in one of the acoustic openings with an interference fit.

In Example 13, the ablation probe of either of Examples 10 or 11, wherein the main cup section is positioned in one of the acoustic openings with the back step extending in a distal direction to provide mechanical retention of the acoustic cup.

In Example 14, the ablation probe of any of Examples 10-13, and further comprising a tip insert having a plurality of recesses each configured for receiving one of the ultrasonic imaging sensors and for partially receiving one of the acoustic cups, wherein each of the plurality of recesses has a recess shoulder upon which the respective acoustic cup rests.

In Example 15, the ablation probe of any of Examples 10-14, wherein the acoustic cups are molded of a polyether block amide.

In Example 16, an ablation probe for treating and imaging body tissue, the ablation probe comprising an ablation electrode tip and a plurality of ultrasonic imaging sensors. The ablation electrode tip includes an ablation electrode configured for delivering ablation energy to body tissue, and further comprises an electrode shell, a proximal tip insert connected to a proximal end of the electrode shell, a distal tip insert and a plurality of acoustic openings. The distal tip insert is disposed within the electrode shell distally of the proximal tip insert, and the plurality of acoustic openings are disposed through the ablation electrode tip. The plurality of ultrasonic imaging sensors are positioned inside the ablation electrode tip and mounted to the distal tip insert, and are configured to transmit ultrasonic waves through the acoustic openings.

In Example 17, the ablation probe of Example 16, wherein the proximal tip insert has a shoulder extending radially outward from and circumferentially around an outer perimeter of the proximal tip insert and wherein the shoulder abuts a rear edge of the electrode shell.

In Example 18, the ablation probe of either of Examples 16 or 17, wherein the proximal tip insert has a recess on an outer perimeter of the proximal tip insert for receiving a distal end of a steering mechanism for deflecting and steering the ablation probe.

In Example 19, the ablation probe of any of Examples 16-18, wherein the proximal tip insert has a central bore passing through the proximal tip insert that is sized and configured to receive electrical and fluid conduits extending into the ablation electrode tip.

In Example 20, the ablation probe of any of Examples 16-19, further comprising a plurality of acoustic cups each coupled to the ablation electrode tip, each acoustic cup positioned at a location corresponding to one of the acoustic imaging sensors.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a combined ablation and imaging system in accordance with an illustrative embodiment;

FIG. 2 is a perspective view showing the distal section of a first embodiment of the combined ablation and ultrasonic imaging probe of FIG. 1 in greater detail;

FIG. 3 is a cross-sectional view of the ablation electrode tip;

FIG. 4 is a cross-sectional view of the ablation electrode tip along line 4-4 in FIG. 2;

FIG. 5 is a cross-sectional view of the RF electrode along line 5-5 in FIG. 2;

FIG. 6 is a perspective view of the proximal tip insert of FIG. 3;

FIG. 7 is a perspective view of the distal tip insert of FIG. 3;

FIG. 8 is an end view of the distal tip insert of FIG. 7 along line 8-8 in FIG. 7;

FIG. 9 is a cross-sectional view of the distal tip insert along line 9-9 in FIG. 7;

FIG. 10 is a perspective view showing the distal section of a second embodiment of the combined ablation and ultrasonic imaging probe of FIG. 1 in greater detail;

FIG. 11 is a perspective view of the distal section of the combined ablation and ultrasonic imaging probe of FIG. 10, with the proximal tip insert and the electrode tip removed;

FIG. 12 is a perspective view of the distal section of the combined ablation and ultrasonic imaging probe of FIG. 10, with the proximal tip insert, the distal tip insert, and the electrode tip removed;

FIG. 13 is a perspective view of the distal section of the combined ablation and ultrasonic imaging probe of FIG. 10, with the proximal tip insert, the distal tip insert, the electrode tip, the acoustic cups, and distal-facing ultrasonic imaging sensor removed;

FIG. 14 is a perspective view showing the distal section of a third embodiment of the combined ablation and ultrasonic imaging probe of FIG. 1 in greater detail; and

FIG. 15 is a schematic side sectional view of the distal section of the combined ablation and ultrasonic imaging probe of FIG. 14.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

FIG. 1 is a schematic view of a combined ablation and imaging system 10 in accordance with an illustrative embodiment. As shown in FIG. 1, the system 10 includes a combined ablation and ultrasonic imaging probe 12, an RF generator 14, a fluid reservoir and pump 16, and an ultrasonic imaging module 18. The probe 12 comprises an elongate probe body 20 having a proximal section 22 equipped with a handle assembly 24, and a deflectable distal section 26 including an ablation electrode tip 28. The probe body 20 includes an internal cooling fluid lumen 29 fluidly coupled to the fluid reservoir and pump 16, which supplies a cooling fluid, such as saline, through the probe body 20 to a number of irrigation ports 30 in the ablation electrode tip 28. The probe body 20 may further include additional lumens or other tubular elements for supporting electrical conductors, additional fluid lumens, a thermocouple, an insertable stylet, as well as other components. In some embodiments, the probe body 20 comprises flexible plastic tubing with a braided metal mesh to increase the rotational stiffness of the body 20.

In various embodiments, the probe 12 includes one or more pacing/sensing electrodes (e.g., circumferential ring electrodes, not shown) on the probe body 20 near the ablation electrode tip 28 for sensing intrinsic cardiac electrical activity and for providing pacing stimuli. In such embodiments, the system 10 may also include additional equipment (not shown) operatively coupled to the pacing/sensing electrodes for recording electrocardiograms and for generating the aforementioned pacing stimuli. Such pacing/sensing components are not critical to the various embodiments, however, and are therefore need not be described in greater detail herein.

The RF generator 14 is configured for generating RF energy for performing ablation procedures using the ablation electrode tip 28. The RF generator 14 includes an RF energy source 32 and a controller 34 for controlling the timing and level of the RF energy delivered by the ablation electrode tip 28. During an ablation procedure, the RF generator 14 is configured to deliver ablation energy to the ablation electrode tip 28 in a controlled manner to ablate any sites identified or targeted for ablation. Other types of ablation sources in addition to or in lieu of the RF generator 14 can also be used for ablating target sites. Examples of other types of ablation sources can include, but are not limited to, microwave generators, acoustic generators, cryoablation generators, and laser/optical generators.

The ultrasonic imaging module 18 is configured for generating high resolution ultrasonic images (e.g., A, M, or B-mode images) of anatomical structures within the body based on signals received from several ultrasonic imaging sensors 36 located within the ablation electrode tip 28. In the embodiment of FIG. 1, the ultrasonic imaging module 18 includes an ultrasonic signal generator 40 and an image processor 42. The ultrasonic signal generator 40 is configured to provide electrical signals for controlling each of the ultrasonic sensors 36. The imaging signals received back from the ultrasonic imaging sensors 36, in turn, are fed to the image processor 42, which processes the signals and generates images that can be displayed on a graphical user interface (GUI) 44. In certain embodiments, for example, the ultrasonic images displayed on the GUI 44 can be used to assist the physician with advancing the probe 12 through the body and to perform an ablation procedure. In cardiac ablation procedures, for example, the ultrasonic images generated from the ultrasound signals can be used to confirm tissue contact of the probe 12 within the heart or surrounding anatomy, to determine the orientation of the probe 12 within the body, to determine the tissue depth of the tissue at a target ablation site, and/or to visualize the progression of a lesion being formed in the tissue.

Various characteristics associated with the ultrasonic imaging sensors 36 as well as the circuitry within the ultrasonic imaging module 18 can be controlled to permit the sensors 36 to accurately detect tissue boundaries (e.g., blood or other bodily fluids), lesion formation and progression, as well as other characteristics of the tissue before, during, and/or after the ablation procedure. Example tissue characteristics that can be visualized using the probe 12 include, but are not limited to, the presence of fluid vaporization inside the tissue, the existence of a prior scar, the size and shape of a lesion being formed, as well as structures adjacent to heart tissue (e.g., lungs, esophagus). The depth at which the ultrasonic imaging sensors 36 can visualize anatomical structures within the body is dependent on the mechanical characteristics of the sensors 36, the electrical characteristics of the sensor circuitry including the drive frequency of the signal generator 40, the boundary conditions and degree of attenuation between the sensors 36 and the surrounding anatomy, as well as other factors.

In some embodiments, the probe 12 further includes a steering mechanism to permit the operator to deflect and steer the probe 12 within the body. In one embodiment, for example, a steering member such as a steering knob 46 rotatably coupled to the handle 24 can be used to deflect the ablation electrode tip 28 in one or multiple directions relative to a longitudinal axis of the probe body 20. Rotational movement of the steering knob 46 in a first direction relative to the handle 24 causes a steering wire within the probe body 20 to move proximally relative to the probe body 20, which, in turn, bends the distal section 26 of the probe body 20 into a particular shape such as an arced shape. Rotational movement of the steering knob 46 in the opposite direction, in turn, causes the distal section 26 of the probe body 20 to return to its original shape, as shown. To assist in the deflection, and in some embodiments, the probe body 20 includes one or more regions made of a lower durometer material than the other portions of the probe body 20.

Although the system 10 is described in the context of a medical system for use in intracardiac electrophysiology procedures for diagnosing and treating the heart, in other embodiments the system 10 may be used for treating, diagnosing, or otherwise visualizing other anatomical structures such as the prostate, brain, gall bladder, uterus, esophagus, and/or other regions in the body. Moreover, many of the elements in FIG. 1 are functional in nature, and are not meant to limit the structure that performs these functions in any manner. For example, several of the functional blocks can be embodied in a single device or one or more of the functional blocks can be embodied in multiple devices.

FIG. 2 is a perspective view showing the distal section 26 of the probe 12 of FIG. 1 in greater detail. As can be further seen in FIG. 2, the ablation electrode tip 28 includes an RF electrode 48 configured for delivering ablation energy to body tissue surrounding the ablation electrode tip 28. In the embodiment of FIG. 2, the RF electrode 48 comprises a tubular-shaped metal electrode shell that extends from a distal end 50 of the probe body 20 to a distal end 52 of the ablation electrode tip 28 along longitudinal axis L. A number of exposed openings 54a, 54b, 54c disposed through the ablation electrode tip 28 form acoustic openings that permit ultrasonic waves transmitted by the ultrasonic imaging sensors 36a, 36b, 36c, 36d to pass through the ablation electrode tip 28 and into the surrounding tissue. The reflected ultrasonic waves received back from the tissue pass through the acoustic openings 54a, 54b, 54c and are sensed by the ultrasonic imaging sensors 36a, 36b, 36c, 36d operating in a receive mode. In some embodiments, the acoustic openings 54a, 54b, 54c comprise exposed openings or apertures formed through the wall of the ablation electrode tip 28.

In addition to serving as an ablation electrode, the RF electrode 48 also functions as a housing that contains the ultrasonic imaging sensors 36a, 36b, 36c, 36d, the electrical conductors coupling the RF electrode 48 to the RF generator 14, the electrical conductors coupling the ultrasonic imaging sensors 36a, 36b, 36c, 36d to the ultrasonic imaging module 18, one or more steering wires of the steering mechanism, as well as other components. In certain embodiments, the RF electrode 48 comprises an electrically conductive alloy such as platinum-iridium, which in addition to serving as an electrode for providing ablation therapy, is also used as a fluoroscopic marker to determine the location of the ablation electrode tip 28 within the body using fluoroscopy.

In the embodiment of FIG. 2, the probe 12 includes a distal-facing ultrasonic imaging sensor 36a located at or near the distal end 52 of the ablation electrode tip 28. In other embodiments, multiple distal-facing ultrasonic imaging sensors 36a are located at or near the distal end 52 of the ablation electrode tip 28. Each ultrasonic sensor 36a is configured to transmit ultrasonic waves primarily in a forward or distal direction away from the distal end 52 of the ablation electrode tip 28. A second set of ultrasonic imaging sensors 36b, 36c, 36d disposed within the ablation electrode tip 28 at a location proximal to the distal-facing ultrasonic imaging sensor 36a are configured to transmit ultrasonic waves primarily in a lateral or side-facing direction away from the side of the ablation electrode tip 28. The reflected waves received back from the ultrasonic imaging sensors 36a, 36b, 36c, 36d produces signals that can be used by the ultrasonic imaging module 18 to generate images of the surrounding body tissue.

In some embodiments, the ultrasonic imaging sensors 36a, 36b, 36c, 36d each comprise piezoelectric transducers formed of a piezoceramic material such as lead zirconate titanate (PZT) or a piezoelectric polymer such as polyvinylidene fluoride (PVDF). In some embodiments, the ablation electrode tip 28 includes three laterally-facing ultrasonic imaging sensors 36b, 36c, 36d each oriented circumferentially at 120° intervals apart from each other about the ablation electrode tip 28 for use in imaging tissue located adjacent to the sides of the ablation electrode tip 28. In other embodiments, a greater or lesser number of laterally-facing ultrasonic imaging sensors are employed for imaging tissue adjacent to the sides of the ablation electrode tip 28.

In the embodiment of FIG. 2, the ablation electrode tip 28 has an open irrigated configuration including a number of irrigation ports 30 used to deliver cooling fluid to cool the ablation electrode tip 28 and the surrounding tissue. In other embodiments, the ablation electrode tip 28 has a closed irrigation configuration in which the cooling fluid is recirculated through the ablation electrode tip 28 without being ejected into the surrounding tissue. In some embodiments, the ablation electrode tip 28 comprises six irrigation ports 30 each disposed circumferentially at 60° intervals apart from each other about the ablation electrode tip 28 and at a location proximal to the distal-facing ultrasonic sensor 36a and distal to the location of the laterally-facing ultrasonic sensors 36b, 36c, 36d. In other embodiments, a greater or lesser number of fluid irrigation ports 30 are employed. In some embodiments, the fluid irrigation ports 30 are circular in shape, and have a diameter in the range of approximately 0.005 inches to 0.02 inches. The size, number, and/or positioning of the irrigation ports 30 can vary, however. In some embodiments, for example, the ablation electrode tip 28 further includes a number of fluid irrigation ports 30 located circumferentially about the ablation electrode tip 28 proximally of the laterally-facing ultrasonic imaging sensors 36b, 36c, 36d. During ablation therapy, the cooling fluid is used to control the temperature and reduce coagulum formation on the ablation electrode tip 28, thus preventing an impedance rise of the tissue in contact with the ablation electrode tip 28 and increasing the transfer of RF ablation energy delivered from the ablation electrode tip 28 into the tissue.

FIG. 3 is a cross-sectional view of the ablation electrode tip 28. As can be further seen in FIG. 3, the ablation electrode tip 28 includes an interior lumen 56 that houses the ultrasonic imaging sensors 36a, 36b, 36c, 36d, electrical conduits 58, 60, 62, 63 for transmitting power to and receiving signals back from the sensors 36a, 36b, 36c, 36d, and an electrical conduit 64 for supplying RF ablation energy to the RF electrode 48. A fluid conduit 66 extending through the probe 12 supplies cooling fluid from the fluid reservoir and pump 16 to the interior lumen 56 of the ablation electrode tip 28, which is then transmitted into the surrounding tissue through the irrigation ports 30. A thermocouple lead 68 extending through the probe 12 terminates distally at a thermocouple 70 located within the interior lumen 56 for sensing the temperature of the ablation electrode tip 28 during the ablation procedure.

A proximal tip insert 72 is used for coupling the ablation electrode tip 28 to the distal end 50 of the probe body 20. A distal tip insert 74 is configured to support the laterally-facing ultrasonic imaging sensors 36b, 36c, 36d within the ablation electrode tip 28, and divides the interior lumen 56 into a proximal fluid chamber 76 and a distal fluid chamber 78. A number of fluid channels 80 extending lengthwise along the length of the distal tip insert 74 fluidly connect the proximal fluid chamber 76 to the distal fluid chamber 78. During ablation, the presence of the distal tip insert 74 within the ablation electrode tip 28 creates a back pressure as the cooling fluid enters the proximal fluid chamber 76, causing the fluid to circulate before being forced through the channels 80 and into the distal fluid chamber 78.

FIG. 4 is a cross-sectional view of the ablation electrode tip 28 along line 4-4 in FIG. 3. As can be further seen in conjunction with FIG. 4, and in some embodiments, the distal tip insert 74 includes three fluid channels 80 for supplying cooling fluid from the proximal fluid chamber 76 to the distal fluid chamber 78. As can be further seen in FIG. 4, and in some embodiments, the ablation electrode tip 28 includes three laterally-facing ultrasonic imaging sensors 36b, 36c, 36d equally spaced from each other at an angle α of 120° about the circumference of the distal tip insert 74. Although three laterally-facing ultrasonic sensors 36b, 36c, 36d are shown in the embodiment of FIG. 4, a greater or lesser number of ultrasonic imaging sensors may be employed. By way of example and not limitation, four ultrasonic imaging sensors may be disposed at equidistant angles α of 90° about the circumference of the distal tip insert 74. During imaging, the use of multiple laterally-facing ultrasonic imaging sensors 36b, 36c, 36d spaced about the circumference of the distal tip insert 74 ensures that the field of view of at least one of the sensors 36b, 36c, 36d is in close proximity to the target tissue irrespective of the tip orientation relative to the target tissue. Such configuration also permits the physician to easily visualize the target tissue without having to rotate the probe 12 once the probe 12 is in contact with the tissue.

To conserve space within the ablation electrode tip 28, the fluid channels 80 are each circumferentially offset from the ultrasonic imaging sensors 36b, 36c, 36d. In the embodiment shown in which three laterally-facing ultrasonic imaging sensors 36b, 36c, 36d are employed, each of the fluid channels 80 are disposed circumferentially at equidistant angles β₁ of 120° about the circumference of the distal tip insert 74, and are circumferentially offset from each adjacent ultrasonic imaging sensor by an angle β₂ of approximately 60°. The angle β₁ between each of the fluid channels 80 and the angle 62 between each fluid channel 80 and adjacent ultrasonic imaging sensors 36b, 36c, 36d can vary in other embodiments depending on the number of fluid channels and/or ultrasonic imaging sensors provided. In some embodiments, the fluid channels 80 each have an equal cross-sectional area and are equally positioned around the center of the distal tip insert 74. The number and configuration of the fluid channels can vary.

FIG. 5 is a cross-sectional view of the RF electrode 48 along line 5-5 in FIG. 2. As can be further seen in FIG. 5, the RF electrode 48 comprises a tubular-shaped electrode shell 82 including six irrigation ports 30 equally spaced from each other at an angle φ of 60° about the circumference of the electrode shell 82. The number, size, and angle φ between each of the irrigation ports 30 can vary in other embodiments. To minimize interference of the irrigation fluid with the transmission of ultrasonic waves from the ultrasonic imaging sensors 36, and in some embodiments, the centers of the irrigation ports 30 are offset circumferentially from the centers of the side-facing acoustic openings 54b, 54c. In those embodiments in which the ablation electrode tip 28 includes three lateral-facing ultrasonic imaging sensors 36b, 36c, 36d and six irrigation ports 30, for example, the irrigation ports 30 can be circumferentially offset from each adjacent side acoustic opening 54b, 54c by an angle of approximately 30°. This circumferential offset may vary in other embodiments depending on the number and configuration of imaging sensors 36 as well as other factors. In some embodiments, the irrigation ports 30 are circular in shape, and have a diameter within a range of approximately 0.005 to 0.02 inches.

FIG. 6 is a perspective view of the proximal tip insert 72 of FIG. 3. As can be further seen in FIG. 6, the proximal tip insert 72 comprises a hollow metal insert body 84 having a proximal section 86 and a distal section 88. The proximal section 86 is configured to attach to the distal end 50 of the probe body 20. The distal section 88, in turn, has an enlarged outer diameter relative to the proximal section 86, and is configured to attach to the electrode shell 82. In some embodiments, the proximal tip insert 72 is coupled to both the distal end 50 of the probe body 20 and to the electrode shell 82 via frictional fit, solder, welding (e.g., laser welding), and/or an adhesive attachment. A shoulder 90 at the transition from the proximal section 86 to the distal section 88 serves as a flange to align the distal end 50 of the probe body 20 flush with the electrode shell 82.

A first lumen 92 disposed through the proximal tip insert 72 provides a conduit for the electrical and fluid conduits 58, 60, 62, 64, 66 that supply electrical signals and cooling fluid to the ablation electrode tip 28. A second lumen 94 disposed through the proximal tip insert 72 provides a conduit for the steering mechanism used for deflecting the probe 12.

FIG. 7 is a perspective view of the distal tip insert 74 of FIG. 3. As shown in FIG. 7, the distal tip insert 74 comprises a cylindrically-shaped metal body 98 having a proximal section 100 and a distal section 102. In the embodiment of FIG. 7, the outer extent 104 of the proximal section 100 is sized to fit within the electrode shell 82 adjacent to the location of the side acoustic openings 54b, 54c, and includes three fluid channels 80. The outer extent 104 further includes a number of recesses 106 each configured to receive a corresponding one of the lateral-facing ultrasonic imaging sensors 36b, 36c, 36d therein. In some embodiments, the recesses 106 are sized and shaped to receive the ultrasonic imaging sensors 36b, 36c, 36d such that the sensors 36b, 36c, 36d lie substantially flush with the outer extent 104. An exposed opening 108 located at the proximal end of the distal tip insert 74 provides a channel to feed the electrical conduits for the ultrasonic imaging sensors 36b, 36c, 36d into the recesses 106.

The distal section 102 of the distal tip insert 74 is configured to support the distal-facing ultrasonic imaging sensor 36a within the ablation electrode tip 28. The outer extent 110 of the distal section 102 is reduced in diameter relative to the proximal section 100. This reduction in diameter creates an annular-shaped distal fluid chamber 78 (see FIG. 3) that receives cooling fluid via the fluid channels 80.

An aperture 112 within the proximal section 100 of the insert body 98 is configured to receive the distal end of a thermocouple used for sensing the temperature of the ablation electrode tip 28. As can be further seen in FIGS. 8-9, a second, central bore 114 extending through the proximal and distal sections 108, 110 of the insert body 104 is configure to receive the distal-facing ultrasonic imaging sensor 36a and a portion of the electrical conduit 63 that connects the sensor 36a to the ultrasonic imaging module 18. In some embodiments, a number of side apertures 116 disposed through the distal section 102 are used to permit alignment and mounting of the distal-facing ultrasonic imaging sensor 36a.

FIG. 10 is a perspective view showing a distal section 26′ of the probe 12 of FIG. 1. The distal section 26′ is an alternative embodiment of the distal section 26 (shown in FIGS. 1 and 2), which both include the electrode shell 82, the proximal tip insert 72, the distal tip insert 74, and the ultrasonic imaging sensors 36a, 36b, 36c, and 36d. The distal section 26′ can be connected to the electrical conduits 60, 62, and 64 as shown in FIG. 10 as well as the electrical conduits 58 and 63, the fluid conduit 66, and the thermocouple lead 68, which are omitted from FIG. 10 for clarity.

The distal section 26′ further includes flex circuits 200, 202, and 204, which are positioned radially inward of and substantially inside the electrode shell 82 and the proximal tip insert 72. In the illustrated embodiment, the flex circuits 200, 202, and 204 terminate inside the proximal tip insert 72 such that the flex circuits 200, 202, and 204 do not extend out of the proximal tip insert 72 in a proximal direction. The ultrasonic imaging sensors 36b, 36c, and 36d are each mounted on and structurally supported by the flex circuits 200, 202, and 204, respectively. The ultrasonic imaging sensors 36b, 36c, and 36d are each also electrically connected to the flex circuits 200, 202, and 204, respectively. In the illustrated embodiment, the ultrasonic imaging sensors 36b, 36c, and 36d have a substantially hexagonal shape.

The distal section 26′ further includes acoustic cups 206 and 208. The acoustic cup 206 is positioned in the side-facing acoustic opening 54b to cover the ultrasonic imaging sensor 36b. The acoustic cup 206 is sized and shaped to substantially fill the side-facing acoustic opening 54b. The acoustic cup 206 has a contoured outer surface that forms a curve that is substantially continuous with that of the cylindrical outer surface of the electrode shell 82. The acoustic cup 206 can allow ultrasonic waves to pass to and from the ultrasonic imaging sensor 36b.

Similarly, the acoustic cup 208 is positioned in the side-facing acoustic opening 54c to cover the ultrasonic imaging sensor 36c. The acoustic cup 208 is sized and shaped to substantially fill the side-facing acoustic opening 54c. The acoustic cup 208 has a contoured outer surface that forms a curve that is substantially continuous with that of the cylindrical outer surface of the electrode shell 82. The acoustic cup 208 can allow ultrasonic waves to pass to and from the ultrasonic imaging sensor 36c.

Although not illustrated in FIG. 10, an additional acoustic cup is also positioned over the radially-facing ultrasonic imaging sensor 36d.

FIG. 11 is a perspective view showing the distal section 26′ with the electrode shell 82 and the proximal tip insert 72 removed for clarity. The flex circuits 200 and 202 are seated partially in the recesses 106, with the lateral-facing ultrasonic imaging sensors 36b and 36c being mounted on and positioned radially outward of the flex circuits 200 and 202, respectively. The acoustic cups 206 and 208 are also seated partially in the recesses 106, on top of and radially outward of both the flex circuits 200 and 202 and the lateral-facing ultrasonic imaging sensors 36b and 36c.

The recesses 106 each have a recess bottom 210 and a recess shoulder 212 positioned radially outward from the recess bottom 210. The flex circuits 200 and 202 each rest on the recess bottoms 210 and the acoustic cups 206 and 208 each rest on the recess shoulders 212.

The flex circuit 200 is a flexible printed circuit having a straight section 211, a straight section 214, and a mounting section 216. A bend 218 is between the straight sections 211 and 214 and another bend 220 is between the straight section 214 and the mounting section 216. The straight section 212 is substantially parallel to, adjacent, and positioned between the electrical conduits 62 and 64. The straight section 211 is angled away from the electrical conduit 62. The mounting section 216 is also substantially parallel to the electrical conduits 62 and 64, but is spaced from the electrical conduit 62 by the distal tip insert 74.

The flex circuit 202 is also a flexible printed circuit having a straight section 222, a straight section 224, and a mounting section 226. A bend 228 is between the straight sections 222 and 224 and another bend 230 is between the straight section 224 and the mounting section 226. The straight section 222 is substantially parallel to, adjacent, and positioned between the electrical conduits 60 and 62. The straight section 224 is angled away from the electrical conduit 62. The mounting section 226 is also substantially parallel to the electrical conduits 60 and 62, but is spaced from the electrical conduit 62 by the distal tip insert 74.

As will be appreciated, the flex circuit 204 can have substantially the same configuration as the flex circuits 200, 202, and can be electrically coupled to the electrical conduit 58 in a similar fashion.

In various embodiments, the flex circuits 200, 202, 204 can be multi-layer, flexible circuits formed from conventional techniques. In one embodiment, the flex circuits 200, 202, 204 each include a structural substrate layer (which can be made of a conductive or non-conductive material) upon which is formed one or more alternating layers of conductive and dielectric layers. In such embodiments, the conductive layer(s) forming one or more conductive traces to facilitate electrical connection of the ultrasonic imaging sensors 36a, 36b, 36c to respective electrical contacts at the proximal end of the probe, and the dielectric layer(s) operate to electrically insulate the conductive trace(s) from one another (if more than one circuit is present) and from other electrically conductive components in the probe.

As shown, the ultrasonic imaging sensor 36b is mounted on the mounting section 216 of the flex circuit 200. In one embodiment, the electrical conduit 64 is a coaxial cable including a core 232, a shield 234, and an insulating sheath 236. Though not illustrated in FIG. 11, the core 232 can be electrically connected to a first electrode (not shown) of the ultrasonic imaging sensor 36b via an electrical trace (not shown) extending along the flex circuit 200 from the straight section 211 to the ultrasonic imaging sensor 36b. The shield 234 can be electrically connected to a second electrode (not shown) of the ultrasonic imaging sensor 36b, for example via a conducting layer (not shown) sputtered on top of the flex circuit 200. Thus, the flex circuit 200 electrically connects the electrical conduit 64 to the ultrasonic imaging sensor 36b for transmitting signals to and from the ultrasonic imaging sensor 36b.

In one embodiment, the ultrasonic imaging sensor 36c is mounted on the mounting section 226 of the flex circuit 202. The electrical conduit 60 is also a coaxial cable including a core 238, a shield 240, and an insulating sheath 242. Though not illustrated in FIG. 11, the core 238 can be electrically connected to a first electrode (not shown) of the ultrasonic imaging sensor 36c via an electrical trace (not shown) extending along the flex circuit 202 from the straight section 222 to the ultrasonic imaging sensor 36c. The shield 240 can be electrically connected to a second electrode (not shown) of the ultrasonic imaging sensor 36c, for example via a conducting layer (not shown) sputtered on top of the flex circuit 202. Thus, the flex circuit 202 electrically connects the electrical conduit 60 to the ultrasonic imaging sensor 36c for transmitting signals to and from the ultrasonic imaging sensor 36c.

FIG. 12 is a perspective view showing the distal section 26′ with the electrode shell 82, the proximal tip insert 72, and the distal tip 74 removed for clarity. As shown in FIG. 12, the flex circuits 200, 202, and 204 are three separate and distinct flex circuits that can combine to effectively form an elongated triangular tube 244 along the straight sections 211 and 222. The triangular tube 244 formed by the flex circuits 200, 202, and 204 can create a channel through which the electrical conduit 62 (and or other conduits) can pass, as well as to create structural rigidity for the distal section 26′.

In one embodiment, the acoustic cup 206 can be a micro-molded component that includes a main cup section 246 and a back step 248 extending from the side of the main cup section 246. The main cup section 246 includes a curved outer surface 250, which faces radially outward from the distal section 26′ with respect to longitudinal axis L (shown in FIG. 2). A substantially cylindrical rim 252 extends radially inward from the outer surface 250. The outer surface 250 and the cylindrical rim 252 combine to form a cup shape within which the ultrasonic imaging sensor 36b is contained. The back step 248 includes an outer surface 254 and side surfaces 256 and 258 extending radially inward from the outer surface 254. The outer surface 250 of the main cup section 246 is radially outward of and axially distal from the outer surface 254 of the back step 248.

Similarly, in one embodiment, the acoustic cup 208 can be a micro-molded component that includes a main cup section 260 and a back step 262 extending from the side of the main cup section 260. The main cup section 260 includes a curved outer surface 264, which faces radially outward from the distal section 26′ with respect to longitudinal axis L. A substantially cylindrical rim 266 extends radially inward from the outer surface 264. The outer surface 264 and the cylindrical rim 266 combine to form a cup shape within which the ultrasonic imaging sensor 36c is contained. The back step 262 includes an outer surface 268 and side surfaces 270 and 272 extending radially inward from the outer surface 268. The outer surface 264 of the main cup section 260 is radially outward of and axially distal from the outer surface 268 of the back step 262.

An acoustic window (not shown) can be used with the ultrasonic imaging sensor 36a, and may or may not be cup-shaped.

FIG. 13 is a perspective view of the distal section 26′, showing only the ultrasonic imaging sensors 36b and 36c, the flex circuits 200, 202, and 204, and the electrical conduits 58, 60, and 64. Although the ultrasonic imaging sensors 36b and 36c are shown in FIG. 13 as mounted on the flex circuits 200 and 202 without the distal tip insert 74 or the electrode shell 82 for illustration purposes, such components can be assembled in a different order.

In one embodiment, the ultrasonic imaging sensors 36b, 36c, 36d can be pre-assembled and mounted to the respective flex circuits 200, 202, 204 and the respective electrical conduits 64, 60, 58 can further be pre-assembled to the flex circuits and ultrasonic imaging sensors for subsequent mounting to the distal tip insert 74.

In one embodiment, the flex circuits 200 and 202 (as well as 204) can be installed inside the electrode shell 82 initially without the ultrasonic imaging sensors 36b and 36c. The ultrasonic imaging sensors 36b and 36c can then be inserted through the side-facing acoustic openings 54b and 54c and soldered onto the flex circuits 200 and 202, respectively.

The acoustic cups 206 and 208 can then be inserted through the respective side-facing acoustic openings 54b and 54c by first inserting the back steps 248 and 262, and then pressing in the main cup sections 246 and 260. The main cup sections 246 and 260 can be configured to be sufficiently resilient to allow it to be press-fit into the side-facing acoustic openings 54b and 54c and thereby held in place via an interference fit, and the back steps 248 and 262 can provide further mechanical retention for the acoustic cups 206 and 208.

An adhesive can be applied between the acoustic cups 206 and 208 and their respective ultrasonic imaging sensors 36b and 36c. The adhesive used to attach the acoustic cups 206 and 208 to the ultrasonic imaging sensors 36b and 36c can be a multipurpose catheter adhesive capable of bonding plastics to metals and capable of transmitting ultrasound, such as an adhesive known as Dymax 209. The acoustic cups 206 and 208 can be transparent or translucent, allowing the adhesive to be cured by ultraviolet light, for example. The acoustic cups 206 and 208 can be made of a material that is suitable for transmitting ultrasound with minimal losses. In various embodiments, the acoustic cups 206, 208 may be made of a material that has an acoustic impedance comparable to that of the surrounding blood or other fluid. In various embodiments, the material of the acoustic cups 206, 208 may have a relatively low hardness such that it can be molded relatively easily. In various embodiments, the material of the acoustic cups 206, 208 may be a polymeric material such as a polyether block amide material, such as those sold under the brand name PEBAX. In various embodiments, a suitable material is a plasticizer-free thermoplastic elastomer such as a PEBAX 5533. In other embodiments, other materials having desired acoustic, mechanical and manufacturability characteristics may be utilized for the acoustic cups 206, 208.

The materials used for the adhesive and the acoustic cups 206 and 208 can facilitate suitable transmission of acoustic waves to and from the ultrasonic imaging sensors 36b and 36c. In alternative embodiments, the acoustic cups 206 and 208 and the adhesive can be made of alternative materials suitable for the application.

FIG. 14 is a perspective view showing a distal section 26″ of the probe 12 of FIG. 1. The distal section 26″ is an alternative embodiment of the distal section 26 (shown in FIGS. 1 and 2) and the distal section 26′ (shown in FIG. 10). The distal section 26″ is similar to the distal section 26′ of FIG. 10 except the distal section 26″ has a proximal tip insert 72″ with a shoulder 300 and a recess 302, both on an outer perimeter 304 of the proximal tip insert 72″.

The shoulder 300 extends radially outward from and circumferentially around the outer perimeter 304 of the proximal tip insert 72″. The shoulder 300 has a diameter substantially equal to that of the electrode shell 82 such that the shoulder 300 abuts a rear edge 308 of the electrode shell 82 when the distal section 26″ is assembled. The shoulder 300 can be attached to the RF electrode shell 82 via an adhesive, solder, or welding.

The recess 302 is an elongated depression on an outer perimeter of the proximal tip insert 72″. The recess 302 has a curved distal end 310 that is positioned proximate the shoulder 300 and has an open proximal end 312 at a proximal edge 314 of the proximal tip insert 72″. The recess 302 is sized and shaped for receiving a steering mechanism (not shown) used to deflect and steer the probe 12 (shown in FIG. 1). The steering mechanism can be attached to the proximal tip insert 72″ at the recess 302 to rigidly connect a distal end of the steering mechanism for deflecting and steering the probe 12.

FIG. 15 is a schematic side sectional view of the distal section 26″. FIG. 15 shows the shoulder 300 abutting the rear edge 308 of the electrode shell 82, as described above. FIG. 15 also shows the ultrasonic imaging sensor 36b and the flex circuit 200 seated in the recess 106. The acoustic cup 206 covers the ultrasonic imaging sensor 36b, with the main cup section 246 having a relatively tight fit inside the side-facing acoustic opening 54b. The back step 248 extends from the main cup section 246 in the distal direction to help retain the acoustic cup 206 in the electrode shell 82.

Although the sectional view of FIG. 15 illustrates only the flex circuit 200 and the electrical conduits 62 and 64 passing through a central bore 316 of the proximal tip insert 72″, the central bore 316 is sized such that all electrical and fluid conduits can be centrally located and pass through the central bore 316. This can provide an electromagnetic shielding function for such conduits, thereby minimizing interference caused by the RF energy supplied during the ablation operation. The flex circuit 200 is shown as having its proximal end 318 terminating within the central bore 316. The flex circuits 202 and 204 (not shown in FIG. 15) can also have their proximal ends terminate within the central bore 316. By terminating the flex circuits 200, 202, and 204 within the central bore 316, exposure to acoustic noise can be reduced.

An acoustic window 320 is positioned in the acoustic opening 54a, adjacent the ultrasonic imaging sensor 36a. The acoustic window 320 can have similar properties and be made of similar materials to those of the acoustic cups 206 and 208.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An ablation probe for treating and imaging body tissue, the ablation probe comprising:
an ablation electrode tip including an ablation electrode configured for delivering ablation energy to body tissue;
an ultrasonic imaging sensor disposed within the ablation electrode tip, the ultrasonic imaging sensor configured to transmit and receive ultrasonic waves; and
a flex circuit mechanically and electrically connected to the ultrasonic imaging sensor.

2. The ablation probe of claim 1, further comprising:
a plurality of ultrasonic imaging sensors disposed within the ablation electrode tip, each of the plurality of ultrasonic imaging sensors configured to transmit and receive ultrasonic waves; and
a plurality of flex circuits, each being mechanically and electrically connected to one of the plurality of ultrasonic imaging sensors.

3. The ablation probe of claim 2, and further comprising:
a plurality of electrical conduits, each electrically connected to one of the plurality of ultrasonic imaging sensors via one of the plurality of flex circuits.

4. The ablation probe of claim 2, wherein the plurality of ultrasonic imaging sensors comprises at least three ultrasonic imaging sensors, wherein the plurality of flex circuits comprises at least three separate and distinct flex circuits each connected to one of the ultrasonic imaging sensors.

5. The ablation probe of claim 2, wherein the plurality of ultrasonic imaging sensors are each mounted on one of the plurality of flex circuits.

6. The ablation probe of claim 2, wherein each of the plurality of flex circuits has a proximal end terminating within a central bore of the ablation electrode tip.

7. The ablation probe of claim 2, wherein the ablation electrode tip has a tubular electrode shell and a plurality of acoustic openings disposed therein, and wherein each of the ultrasonic imaging sensors is aligned with a respective one of the acoustic openings.

8. The ablation probe of claim 7, wherein the plurality of ultrasonic imaging sensors includes three ultrasonic imaging transducers oriented circumferentially about the ablation electrode tip.

9. The ablation probe of claim 7, wherein the ablation tip further comprises a plurality of irrigation ports formed in the tubular electrode shell distally of the acoustic openings.

10. An ablation probe for treating and imaging body tissue, the ablation probe comprising:
an ablation electrode tip including an ablation electrode configured for delivering ablation energy to body tissue;
a plurality of acoustic openings disposed through the ablation electrode tip;
a plurality of ultrasonic imaging sensors positioned inside the ablation electrode tip, each aligned with one of the acoustic openings; and
a plurality of acoustic cups covering each of the ultrasonic imaging sensors.

11. The ablation probe of claim 10, wherein each of the acoustic cups comprises:
a main cup section; and
a back step extending from a side of the main cup section.

12. The ablation probe of claim 11, wherein the main cup section is positioned in one of the acoustic openings with an interference fit.

13. The ablation probe of claim 11, wherein the main cup section is positioned in one of the acoustic openings with the back step extending in a distal direction to provide mechanical retention of the acoustic cup.

14. The ablation probe of claim 10, and further comprising:
a tip insert having a plurality of recesses each configured for receiving one of the ultrasonic imaging sensors and for partially receiving one of the acoustic cups, wherein each of the plurality of recesses has a recess shoulder upon which the respective acoustic cup rests.

15. The ablation probe of claim 10, wherein the acoustic cups are molded of a polyether block amide.

16. An ablation probe for treating and imaging body tissue, the ablation probe comprising:
an ablation electrode tip including an ablation electrode configured for delivering ablation energy to body tissue, the ablation electrode tip comprising:
an electrode shell;
a proximal tip insert connected to a proximal end of the electrode shell;
a distal tip insert disposed within the electrode shell distally of the proximal tip insert; and
a plurality of acoustic openings disposed through the ablation electrode tip; and
a plurality of ultrasonic imaging sensors positioned inside the ablation electrode tip and mounted to the distal tip insert, the ultrasonic imaging sensors configured to transmit ultrasonic waves through the acoustic openings.

17. The ablation probe of claim 16, wherein the proximal tip insert has a shoulder extending radially outward from and circumferentially around an outer perimeter of the proximal tip insert and wherein the shoulder abuts a rear edge of the electrode shell.

18. The ablation probe of claim 16, wherein the proximal tip insert has a recess on an outer perimeter of the proximal tip insert for receiving a distal end of a steering mechanism for deflecting and steering the ablation probe.

19. The ablation probe of claim 16, wherein the proximal tip insert has a central bore passing through the proximal tip insert that is sized and configured to receive electrical and fluid conduits extending into the ablation electrode tip.

20. The ablation probe of claim 16, further comprising a plurality of acoustic cups each coupled to the ablation electrode tip, each acoustic cup positioned at a location corresponding to one of the acoustic imaging sensors.
